(19) United States
(12) Patent Application Publication
(10) Pub. No.: US 2017/0156589 A1
(43) Pub. Date: Jun. 8, 2017

(54) Method of identification based on smart glasses

(71) Applicant: SHENZHEN UNIVERSITY, Shenzhen (CN)

(72) Inventors: Kaishun WU, Shenzhen (CN); Yongpan ZOU, Shenzhen (CN)

(73) Assignee: SHENZHEN UNIVERSITY, Shenzhen (CN)

(21) Appl. No.: 15/212,196

(22) Filed: Jul. 16, 2016

(30) Foreign Application Priority Data
Dec. 4, 2015 (CN) 201510878498.6

(51) Int. Cl. A61B 3/113; G09B 17/00; G06F 3/01; G06K 9/00; G02B 27/01; G02B 27/00

(52) U.S. Cl. CPC A61B 3/113; G02B 27/0172; G02B 27/0093; G06F 3/012; G06F 3/013; G06K 9/00604; G09B 17/003; G02B 2027/0178; G02B 2027/0138; G02B 2027/014; G02B 2027/0141

(57) ABSTRACT

The present invention relates to the field of smart identification, in particular a method of identification based on smart glasses, comprising the following steps: obtains real-time data transmitted from an inertial sensor on the glasses and determines the head position; obtains reading states and contents; reminder for reading. The beneficial effects of the present invention are as below: it detects and identifies users' cognitive state, and provides users with corresponding cognitive assistant measures, to further improve the reading efficiency.

### BACKGROUND OF THE INVENTION

Technical Field

The present invention relates to the field of smart identification, and more especially, to a method of identification based on smart glasses.

2. Description of Related Art

Nowadays, with the growing popularity of education, people's reading demand is gradually increasing. However, as reading contents become richer and richer, people may encounter more and more problems (for example, people may run into a lot of words and sentences they cannot understand when reading English articles, may run into many professional terms they cannot understand when reading professional books, and may run into plenty of specialized knowledge they cannot understand when reading literature). When facing these problems, people need to search for the relevant information, but it is a very troublesome and time-consuming thing, as it will greatly reduce people's reading efficiency.

To solve people's reading problems and improve people's reading efficiency, existing commercial products, such as the BBK finger reader, almost adopt a finger reading technology, through which the corresponding prompt contents will be displayed when people use a pen to click on a book put in a certain place and to sense a certain position in the book. The technology has many limitations, as it can only be used after recording the contents in a book and then making it into a software package, and ordinary books in the market are incapable of using this finger reader; secondly, it is very inconvenient for people to take along such a large-volume finger reader each time when they are reading; and this kind of finger reader is unable to remind people of returning back to reading, when people tend to become distracted during reading. In this case, a system that can help people overcome the difficulties in reading and make a sound when people become distracted undoubtedly has a high practical value.

### BRIEF SUMMARY OF THE INVENTION

To solve problems in the prior art, the present invention provides a method of identification based on smart glasses, aimed at solving the problem that the prior art is unable to help people overcome difficulties during reading and unable to make a sound when people become distracted.

The present invention is realized by adopting the following technical solution: it designs and provides a method of identification based on smart glasses, comprising the following steps: (S1) obtain real-time data transmitted from an inertial sensor on the glasses and determine the head position; (S2) obtain reading states and contents; (S3) reminder for reading.

As a further improvement of the present invention, Step (S1) further includes: (S11) read data from an inertial sensor on the glasses; (S12) determine people's reading states by judging various head positions; when the head position fits the range of head position during reading and people are not moving, it is deemed as a reading state, and when the head position does not fit the range of head position or people are moving, it is deemed as a not-reading state.

As a further improvement of the present invention, in Step (S2), by converting images transmitted from an eye camera on the glasses into a gray value, it sets a threshold value so as to obtain the coordinates of the eyeballs; by judging the coordinates of the eyeballs as time changes, it obtains people's various reading states; and by setting coordinates of images from a scene camera on the glasses and using a calibrating method, keeps the coordinates of images from the scene camera in one-to-one correspondence with the coordinates of the eyeballs, so as to obtain contents that users are reading now.

As a further improvement of the present invention, in Step (S3), it provides various reading aids for readers according to reading states.

As a further improvement of the present invention, in Step (S2), to obtain various reading states, it further includes the following steps: (S21) turn on an IR emitter and shine it on the eyes, turn on the eye camera to capture eye position images, and turn on the scene camera to capture images that eyes have seen; (S22) by converting images transmitted from an eye camera into a gray value and reversing it, set a threshold value of the gray value so as to obtain the positions of the pupils, and set the coordinates of the pupil centers; (S23) determine people's reading states via eyeball movements.

As a further improvement of the present invention, the reading states include normal reading state, thinking state, glancing state, reviewing state and distracted state; the normal reading state is that the eyeball movement speed remains in a certain range; the thinking state is that the eyeball movement speed remains in a certain range and the time exceeds a threshold value; the glancing state is that the eyeball movement speed exceeds a threshold value; the reviewing state is that the eyeballs move in an opposite direction; and the distracted state is that the eyeball movement speed is lower than a threshold value for more than a period of time.

As a further improvement of the present invention, in Step (S3), it divides images obtained from the scene camera into nine squares and sets coordinates, and uses a calibrating method to keep the coordinates of scene images in one-to-one correspondence with the coordinates of the eyeballs; by obtaining the reading states, it conducts reading instruction.

As a further improvement of the present invention, by multiplying the eyeball coordinate matrix by the matrix calculated based on calibration, it works out coordinates of the scene images.

As a further improvement of the present invention, when the reading state stays in a thinking state, it will automatically search the contents that users are reading and then display the search results; when users are in a distracted state, it will automatically make a sound as a reminder.

The beneficial effects of the present invention are as below: it detects and identifies users' cognitive state, and provides users with corresponding cognitive assistant measures, to further improve the reading efficiency.

### BRIEF DESCRIPTION OF THE SEVERAL VIEWS OF THE DRAWINGS

FIG. 1 is a step diagram of the method of identification based on smart glasses of the present invention.

FIG. 2 is a diagram of an embodiment of the present invention.

### DETAILED DESCRIPTION OF THE INVENTION

The invention is further detailed in combination with the drawings and embodiments as follows.

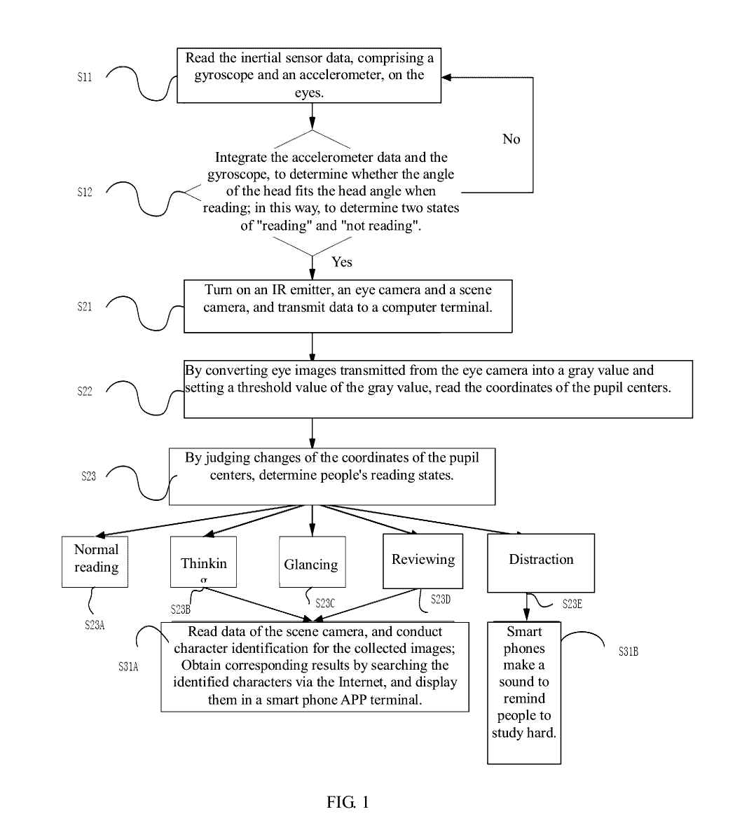

As shown in FIG. 1, a method of identification based on smart glasses, comprising the following steps: (51) obtain real-time data transmitted from an inertial sensor on the glasses and determines the head position; (S2) obtain reading states and contents; (S3) reminder for reading.

The Step (51) further includes: (S11) read data from an inertial sensor (comprising an accelerometer and a gyroscope) on the glasses; (S12) determine people's reading states by judging various head positions; when the head position fits the range of head position during reading and people are not moving, it is deemed as a reading state, and when the head position does not fit the range of head position or people are moving, it is deemed as a not-reading state.

In Step (S2), by converting images transmitted from an eye camera on the glasses into a gray value, set a threshold value so as to obtain the coordinates of the eyeballs; by judging the coordinates of eyeballs as time changes, obtain people's various reading states; and by setting coordinates of images from a scene camera on the glasses and using a calibrating method, keeps the coordinates of images from the scene camera in one-to-one correspondence with the coordinates of the eyeballs, so as to obtain contents that users are reading now.

In Step (S3), provide various reading aids for readers according to reading states.

In Step (S2), to obtain various reading states, it further includes the following steps: (S21) turn on an IR emitter and shine it on the eyes, turn on the eye camera to capture eye position images, and turn on the scene camera to capture images that eyes have seen; (S22) by converting images transmitted from an eye camera into a gray value and reversing it, set a threshold value of the gray value so as to obtain the positions of the pupils, and set the coordinates of the pupil centers (it makes use of the characteristics of eyes to the IR, namely, when the IR and the optical axis stay in different axes, pupils will turn extremely dark while irises will turn relatively bright); (S23) determine people's reading states via eyeball movements.

The reading states include normal reading state, thinking state, glancing state, reviewing state and distracted state; the normal reading state is that the eyeball movement speed remains in a certain range; the thinking state is that the eyeball movement speed remains in a certain range and the time exceeds a threshold value; the glancing state is that the eyeball movement speed exceeds a threshold value; the reviewing state is that the eyeballs move in an opposite direction; and the distracted state is that the eyeball movement speed is lower than a threshold value for more than a period of time.

In Step (S3), it divides images obtained from the scene camera into nine squares and sets coordinates, and uses a calibrating method to keep the coordinates of scene images in one-to-one correspondence with the coordinates of eyeballs; by obtaining the reading states, it conducts reading instruction.

By multiplying the eyeball coordinate matrix by the matrix calculated based on calibration, it works out coordinates of scene images.

When the reading state stays in a thinking state, it will automatically search the contents that users are reading and then display the search results; when users are in a distracted state, it will automatically make a sound as reminder.

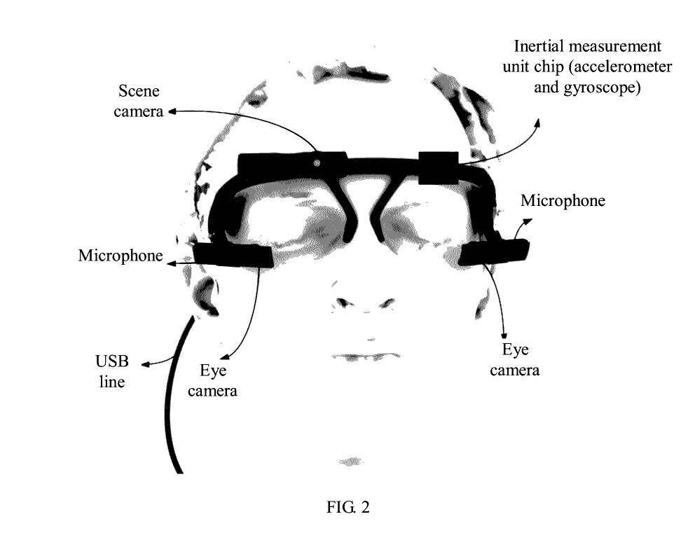

In an embodiment, as shown in FIG. 2, it provides a smart system based on smart glasses in combination with eyeball tracking, image coordinate matching, text identification and machine learning technologies, thus detecting and identifying users' cognitive state, and provides them with corresponding cognitive assistant measures. The method realizes the above-mentioned functions by using a camera arranged on the glasses, an IR emitter and an inertial measurement unit (an accelerometer and a gyroscope), supported by a smart phone APP. The method mainly comprises: obtain users' head movement data via monitoring by a built-in inertial measurement unit; determine users' behavior states via analysis on data from the inertial measurement unit; when it determines that users are in a reading state, the IR emitter, the eyeball camera and the scene camera will be turned on simultaneously, so that on one hand, it obtains images of users' eyeball movement during reading via the IR sensor and eye camera, and on the other hand, it obtains images of users' current reading contents via the scene camera; determines users' learning and cognitive states during reading, such as normal reading, thinking, reviewing, glancing and distraction, by processing images of eyeball movements to extract features of eyeball movements, in combination with data from the inertial measurement unit; and identifies contents in images from the scene camera, by using a calibrating method, keeps the coordinates of the eyeballs in one-to-one correspondence with coordinates of scenes, and analyzes users' learning interests, cognitive features and behavior habits, by identifying contents in images from the scene camera in combination with the determination on users' learning and cognitive states. Based on these core modules, we can develop a variety of learning and cognitive assistant APPs for users; for example, when the system determines that a user is gazing on a certain content for quite a long time, the corresponding APP will automatically search the content that has been identified, to help users understand the corresponding content.

In an embodiment, S1. it determines the head position (head deflecting angles, which include front, rear, left and right sides) by reading data of an accelerometer and a gyroscope, after an inertial sensor on the glasses transmits real-time data to a computer; S2. it determines people's reading states based on head positions, with the states divided into “reading” and “not reading”, and the contents including paper and electronic materials. When people stay in a reading state, the IR emitter, the eyeball camera and the scene camera will be turned on simultaneously and collect data. The collected data are processed via server, PC, smart phone, tablet and other devices. By converting images transmitted from an eye camera into a gray value, it sets a threshold value so as to obtain coordinates of eyeballs. By judging the coordinates of eyeballs as time changes, it obtains people's various reading states, such as normal reading, thinking, glancing, reviewing and distraction. By setting coordinates of images from a scene camera and using a calibrating method, it keeps the coordinates of images from the scene camera in one-to-one correspondence with the coordinates of eyeballs, so as to obtain contents that users are reading now; S3. it provides reading aids for readers according to various reading states obtained in Step S2. For example, when readers stay in a thinking state or review the foregoing contents for many times, a smart phone APP will automatically search the contents that users are reading to provide aids for readers, so as to solve readers' reading problems; when readers stay in a distracted state, the smart phone APP will make a sound to remind people of returning back to reading, so as to improve people's reading efficiency.

Providing various reading aids according to reading states further comprises: it divides images obtained from the scene camera into nine squares and sets coordinates, and uses a calibrating method to keep the coordinates of scene images in one-to-one correspondence with the coordinates of eyeballs. The way of correspondence can be obtained via matrix multiplication. That is to say, by multiplying the eyeball coordinate matrix by the matrix calculated based on calibration, it works out coordinates of scene images. It provides various aids according to the states obtained in Step S2. When the reading state stays in a thinking state, a smart phone APP will automatically search the contents that users are reading and then display the search results on the smart phone APP, to help readers solve problems they encounter during reading; it can help readers improve the reading efficiency according to the states obtained in Step S2, namely, when users stay in a distracted state, the smart phone APP will automatically make a sound to remind readers of reading carefully and not becoming distracted, so as to improve readers' efficiency.

The foregoing are further detailed for the present invention in combination with detailed preferable embodiments, but are not intended to limit detailed embodiments of the present invention. Those skilled in this art can make a variety of simple deductions or variations without deviating from the principle of the present invention, and all these should be covered in the protection scope of the present invention.

1. A method of identification based on smart glasses, characterized in that they comprise the following steps: obtain real-time data transmitted from an inertial sensor on the glasses and determine the head position; obtain the reading states and contents; reminder for reading.

2. The method of identification based on smart glasses as claimed in claim 1, characterized in that further includes: read data from an inertial sensor on the glasses; determine people's reading states by judging various head positions; when the head position fits the range of head position during reading and people are not moving, it is deemed as a reading state, and when the head position does not fit the range of head position or people are moving, it is deemed as a not-reading state.

3. The method of identification based on smart glasses as claimed in claim 1, characterized in that, in Step, by converting images transmitted from an eye camera on the glasses into a gray value, it sets a threshold value so as to obtain the coordinates of the eyeballs; by judging the coordinates of the eyeballs as time changes, it obtains people's various reading states; and by setting coordinates of images from a scene camera on the glasses and using a calibrating method, keeps the coordinates of images from the scene camera in one-to-one correspondence with the coordinates of the eyeballs, so as to obtain contents that users are reading now.

4. The method of identification based on smart glasses as claimed in claim 1, characterized in that, in Step, it provides various reading aids for readers according to reading states.

5. The method of identification based on smart glasses as claimed in claim 3, characterized in that, in Step, to obtain various reading states, it further includes the following steps: turn on an IR emitter and shine it on the eyes, turn on the eye camera to capture eye position images, and turn on the scene camera to capture images that the eyes have seen; by converting images transmitted from an eye camera into a gray value and reversing it, set a threshold value of the gray value so as to obtain the positions of the pupils, and set the coordinates of the pupil centers; determine people's reading states via eyeball movements.

6. The method of identification based on smart glasses as claimed in claim 1, characterized in that the reading states include normal reading state, thinking state, glancing state, reviewing state and distracted state; the normal reading state is that the eyeball movement speed remains in a certain range; the thinking state is that the eyeball movement speed remains in a certain range and the time exceeds a threshold value; the glancing state is that the eyeball movement speed exceeds a threshold value; the reviewing state is that the eyeballs move in an opposite direction; and the distracted state is that the eyeball movement speed is lower than a threshold value for more than a period of time.

7. The method of identification based on smart glasses as claimed in claim 1, characterized in that, in Step, it divides images obtained from the scene camera into nine squares and sets coordinates, and uses a calibrating method to keep the coordinates of scene images in one-to-one correspondence with the coordinates of the eyeballs; by obtaining the reading states, it conducts reading instruction.

8. The method of identification based on smart glasses as claimed in claim 7, characterized in that, by multiplying the eyeball coordinate matrix by the matrix calculated based on calibration, it works out coordinates of scene images.

9. The method of identification based on smart glasses as claimed in claim 6, characterized in that, when the reading state stays in a thinking state, it will automatically search the contents that users are reading and then display the search results; when users are in a distracted state, it will automatically make a sound as reminder.
